(19) United States
(12) Patent Application Publication
(10) Pub. No.: US 2019/0218226 A1
(43) Pub. Date: Jul. 18, 2019

(54) PRODRUGS OF ANTICANCER AGENTS INDOTECAN AND INDIMITECAN

(71) Applicant: Purdue Research Foundation, West Lafayette, IN (US)

(72) Inventors: Mark S. Cushman, West Lafayette, IN (US); Pengcheng LV, Hoover, AL (US)

(21) Appl. No.: 16/075,477

(22) PCT Filed: Feb. 3, 2017
(86) PCT No.: PCT/US2017/016331

§ 371 Date: Aug. 3, 2018

Related U.S. Application Data
(60) Provisional application No. 62/291,292, filed on Feb. 4, 2016.

(51) Int. Cl. C07D 491/056; A61P 35/00

(52) U.S. Cl. CPC C07D 491/056; A61P 35/00

(57) ABSTRACT

Indenoisoquinoline topoisomerase I (Top1) inhibitors are a novel class of anticancer agents. The present invention discloses series of prodrugs of two indenoisoquinoline compounds currently in clinical trials as a potential treatment for cancers.

### CROSS-REFERENCE TO RELATED APPLICATIONS

The present U.S. patent application is related to and claims the priority benefit of U.S. Provisional Patent Application Ser. No. 62/291,292, filed Feb. 4, 2016, the contents of which are hereby incorporated by reference in their entirety into the present disclosure.

### GOVERNMENT RIGHTS

This invention was made with government support under grants CA089566 and CA023168, awarded by the National Institutes of Health. The U.S. government has certain rights in the invention.

### TECHNICAL FIELD

The present invention generally relates to novel compounds as cancer therapeutics, and in particular to prodrugs of anticancer agents indotecan (LMP400) and indimitecan (LMP776). The invention described herein also pertains to methods for treating patients with cancer using those prodrugs.

### BACKGROUND

This section introduces aspects that may help facilitate a better understanding of the disclosure. Accordingly, these statements are to be read in this light and are not to be understood as admissions about what is or is not prior art.

Topoisomerases are ubiquitous enzymes that resolve the topological problems associated with DNA supercoiling during replication, transcription, and other nuclear processes. Human topoisomerase I (Top1) cleaves a single DNA strand by nucleophilic attack of the enzyme on a DNA phosphodiester to form a “cleavage complex” in which the 3′ end of the broken DNA strand is covalently linked to the enzyme (Scheme 1) (Staker, B. L., et al., The Mechanism of Topoisomerase I Poisoning by a Camptothecin Analog. Proc. Natl. Acad. Sci. U.S.A. 2002, 99, 15387-15392). The broken (scissile) strand then undergoes “controlled rotation” around the unbroken strand to relax the DNA superhelical tension and remove supercoils. The catalytic cycle ends when the 5′ end of the scissile strand carries out a nucleophilic attack on the phosphotyrosyl-DNA phosphodiester to religate the DNA and release the enzyme.

Top1 inhibitors are classified as Top1 suppressors, which inhibit the DNA cleavage reaction, and Top1 poisons, which inhibit the DNA religation reaction (Scheme 1). Top1 is overexpressed in cancer cells and DNA damage responses are defective in some human tumors. Top1 poisons that stabilize the “cleavage complex” have therefore been developed as chemotherapeutic agents. The mechanism of cancer cell death produced by Top1 poisons involves collision of the DNA replication fork with the DNA cleavage site in the ternary DNA-drug-Top1 complex leading to double-strand breaks and cell death (Pommier, Y., Nat. Rev. Cancer 2006, 6, 789-802).

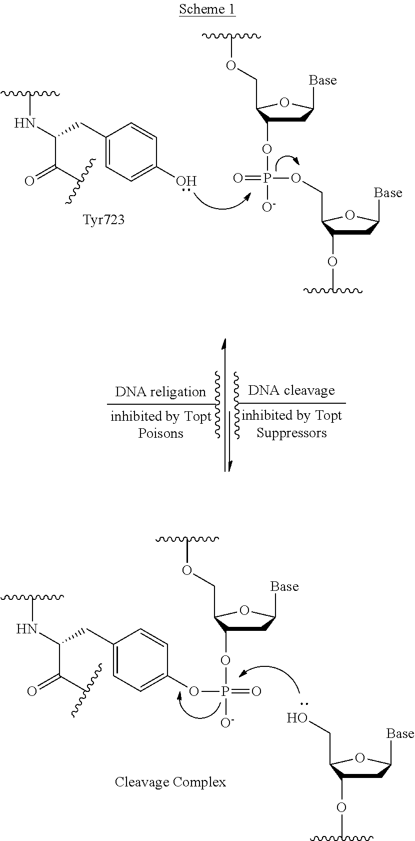

Representative Top1 poisons are shown in FIG. 1 (Wall, M. E., et al., J. Am. Chem. Soc. 1966, 88, 3888-3890). Camptothecin (1) is a natural product having Top1 as its only cellular target. Although topotecan (2) and irinotecan (3) are approved by the Food and Drug Administration (FDA) for the treatment of cancer, camptothecin derivatives suffer from several major drawbacks, including poor aqueous solubility, dose-limiting toxicity, and bioavailability limitations resulting from lactone hydrolysis and binding of the ensuing hydroxyacid to plasma proteins. These limitations have stimulated the search for noncamptothecin Top1 inhibitors as anticancer agents. The Top1 poisoning activity of NSC314622 (4) was discovered after a COMPARE Algorithm analysis (Paull, K. D., et al., J. Natl. Cancer Inst. 1989, 81, 1088-1092) of its cytotoxicity profile revealed a strong resemblance to that of other known Top1 poisons, including camptothecin (1) and the clinically useful anticancer drug topotecan (2).

Subsequently, MJ-III-65 (5) was found to be a potent Top1 poison after a hydroxyethyl-aminopropyl side chain was attached to the lactam nitrogen of 4. The indenoisoquinolines have several advantages over the camptothecins. Two indenoisoquinoline Top1 poisons, indotecan (6, also known as LMP400) and indimitecan (7, also known as LMP776), have entered Phase I clinical trials for treatment of cancer patients at the National Cancer Institute, and definite plans are being formulated to commence Phase II clinical trials (Teicher, B., Biochem. Pharmacol. 2008, 75, 1262-1271; Thomas, C. J., et al., Camptothecin: Current Perspectives. Bioorg. Med. Chem. 2004, 12, 1585-1604; Pommier, Y., et al., Chem. Biol. 2010, 17, 421-433).

Our continuing research efforts in this field have led the discovery of series of prodrugs of indotecan (6, LMP400) and indimitecan (7, LMP776). Prodrugs are bioreversible derivatives of drug molecules that undergo an enzymatic and/or chemical transformation in vivo to release the active parent drug, which can then exert the desired pharmacological effect (Stella, V. J., Expert Opin. On Ther. Pat. 2004, 14, 277-280; Testa, B., Biochem. Pharmacol. 2004, 68, 2097-2106). Irinotecan (3) provides a good example, since the carbamate is hydrolyzed after administration to the pharmacologically active phenol. The prodrug strategy can improve the physicochemical, biopharmaceutical, or pharmacokinetic properties of pharmacologically potent compounds, and thereby enhance the development and usefulness of a potential drug product (Rautio, J. et al. Nat. Rev. Drug Discov. 2008, 7, 255-270; Arpicco, S., et al., Curr. Top. Med. Chem. 2011, 11, 2346-2381).

### BRIEF SUMMARY OF INVENTION

In one embodiment, disclosed herein are compounds having the formula (I):

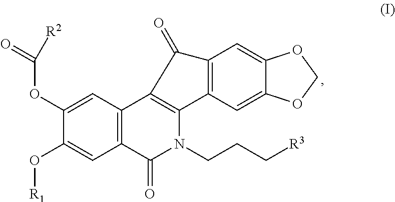

or a pharmaceutically acceptable salt, hydrate, or solvate thereof, wherein

R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;

R² is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₈ cycloalkylamide, C₁-C₆ alkylamide, C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and

R³ is

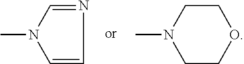

In another embodiment, disclosed herein are compounds having the formula (II):

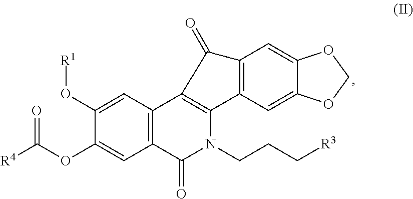

or a pharmaceutically acceptable salt, hydrate, or solvate thereof, wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
R³ is

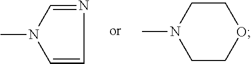

and
R⁴ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₈ cycloalkylamide, C₁-C₆ alkylamide, C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl.

In some embodiments, disclosed herein are compounds having the formula (III):

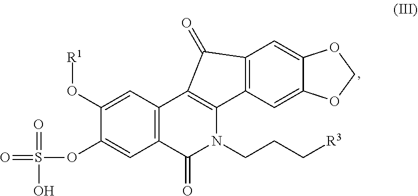

or a pharmaceutically acceptable salt, hydrate, or solvate thereof, wherein

R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;

and

R³ is

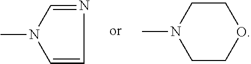

In one embodiment, disclosed herein is a pharmaceutical composition comprising a compound of formula (I), and one or more pharmaceutically acceptable carriers, diluents, and excipients.

In another embodiment, disclosed herein is a pharmaceutical composition comprising a compound of formula (II), and one or more pharmaceutically acceptable carriers, diluents, and excipients.

In another embodiment, disclosed herein is a pharmaceutical composition comprising a compound of formula (III), and one or more pharmaceutically acceptable carriers, diluents, and excipients.

It is to be understood that the pharmaceutical composition of (I), (II), or (III) may be combined with other components, including, but not limited to, other therapeutically active compounds by the same or different mode of action, and one or more pharmaceutically acceptable carriers, diluents, excipients, and the like.

In another embodiment, pharmaceutical compositions described herein may contain two or more of the compounds disclosed in this invention.

In another embodiment, it is appreciated herein that the compounds described herein may be used alone or in combination with other compounds useful for treating cancer, including those compounds that may be therapeutically effective by the same or different mode of action.

In one embodiment, disclosed herein is a method for treating a patient with cancer, the method comprising the step of administering a therapeutically effective amount of a pharmaceutical composition comprising the compound of formula (I) to the patient in need of relief from said cancer. It is to be understood that the composition may include other components, including, but not limited to, other therapeutically active compounds by the same or different mode of action, and one or more pharmaceutically acceptable carriers, diluents, excipients, and the like.

In one embodiment, disclosed herein is a method for treating a patient with cancer, the method comprising the step of administering a therapeutically effective amount of a pharmaceutical composition comprising the compound of formula (II) to the patient in need of relief from said cancer.

In one embodiment, disclosed herein is a method for treating a patient with cancer, the method comprising the step of administering a therapeutically effective amount of a pharmaceutical composition comprising the compound of formula (III) to the patient in need of relief from said cancer.

In another embodiment, disclosed herein is a method for treating a patient with cancer, the method comprising the step of administering a therapeutically effective amount of a compound of the formula (I), (II), or (III), in combination with one or more therapeutically effective compounds by the same or different mode of action to the patient in need of relief from said cancer.

In another embodiment, it is also appreciated herein that the compounds described herein may be used in combination with other compounds that are administered to treat other symptoms of cancer, such as compounds administered to relieve nausea, vomiting, pain, osteoporosis, and the like.

### BRIEF DESCRIPTION OF THE FIGURES

The above and other objects, features, and advantages of the present invention will become more apparent when taken in conjunction with the following description and drawings, wherein:

FIG. 1 provides the structures of representative Top1 poisons; and

FIG. 2 shows the results of Top1-mediated DNA cleavages induced by indenoisoquinolines 20, 14, 10, 13 and 11: lane 1, DNA alone; lane 2, Top1+DNA; lane 3, 1, 1 μM; lane 4, 5, 1 μM; lanes 5-24, 20, 14, 10, 13, and 11 at 0.1, 1, 10, and 100 μM, respectively, from left to right. Numbers and arrows on the left indicate cleavage site positions.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The invention disclosed herein provides novel compounds that are useful for the treatment of a patient with cancer. Those phenol ester compounds are prodrugs of indotecan (LMP400) and indimitecan (LMP776) (Teicher, B., Biochem. Pharmacol. 2008, 75, 1262-1271; Pommier, Y., et al., Chem. Biol. 2010, 17, 421-433).

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, or within 99% of a stated value or of a stated limit of a range.

A “halogen” designates F, Cl, Br or I. A “halogen-substitution” or “halo” substitution designates replacement of one or more hydrogen atoms with F, Cl, Br or I.

As used herein, the term “alkyl” refers to a saturated monovalent chain of carbon atoms, which may be optionally branched. It is understood that in embodiments that include alkyl, illustrative variations of those embodiments include lower alkyl, such as C₁-C₆ alkyl, methyl, ethyl, propyl, 3-methylpentyl, and the like.

As used herein, the term “alkenyl” refers to an unsaturated monovalent chain of carbon atoms including at least one double bond, which may be optionally branched. It is understood that in embodiments that include alkenyl, illustrative variations of those embodiments include lower alkenyl, such as C₂-C₆, C₂-C₄ alkenyl, and the like.

As used herein, the term “alkynyl” refers to an unsaturated monovalent chain of carbon atoms including at least one triple bond, which may be optionally branched. It is understood that in embodiments that include alkynyl, illustrative variations of those embodiments include lower alkynyl, such as C₂-C₆, C₂-C₄ alkynyl, and the like.

As used herein, the term “cycloalkyl” refers to a monovalent chain of carbon atoms, a portion of which forms a ring. It is understood that in embodiments that include cycloalkyl, illustrative variations of those embodiments include lower cycloalkyl, such as C₃-C₈ cycloalkyl, cyclopropyl, cyclohexyl, 3-ethylcyclopentyl, and the like.

As used herein, the term “cycloalkenyl” refers to an unsaturated monovalent chain of carbon atoms, a portion of which forms a ring. It is understood that in embodiments that include cycloalkenyl, illustrative variations of those embodiments include lower cycloalkenyl, such as C₃-C₈, C₃-C₆ cycloalkenyl.

As used herein, the term “alkylene” refers to a saturated bivalent chain of carbon atoms, which may be optionally branched. It is understood that in embodiments that include alkylene, illustrative variations of those embodiments include lower alkylene, such as C₂-C₄, alkylene, methylene, ethylene, propylene, 3-methylpentylene, and the like.

As used herein, the term “heterocyclic” or “heterocycle” refers to a monovalent chain of carbon and heteroatoms, wherein the heteroatoms are selected from nitrogen, oxygen, and sulfur, and a portion of which, at least one heteroatom, forms a ring. The term “heterocycle” may include both “aromatic heterocycles” and “non-aromatic heterocycles.” Heterocycles include 4-7 membered monocyclic and 8-12 membered bicyclic rings, such as imidazolyl, thiazolyl, oxazolyl, oxazinyl, thiazinyl, dithianyl, dioxanyl, isoxazolyl, isothiazolyl, triazolyl, furanyl, tetrahydrofuranyl, dihydrofuranyl, pyranyl, tetrazolyl, pyrazolyl, pyrazinyl, pyridazinyl, imidazolyl, pyridinyl, pyrrolyl, dihydropyrrolyl, pyrrolidinyl, piperidinyl, piperazinyl, pyrimidinyl, morpholinyl, tetrahydrothiophenyl, thiophenyl, azetidinyl, oxetanyl, thiiranyl, oxiranyl, aziridinyl, and the like. “Heterocycles” may be optionally substituted at any one or more positions capable of bearing a hydrogen atom.

As used herein, the term “aryl” includes monocyclic and polycyclic aromatic carbocyclic groups, each of which may be optionally substituted. The term “optionally substituted aryl” refers to an aromatic mono or polycyclic ring of carbon atoms, such as phenyl, naphthyl, and the like, which may be optionally substituted with one or more independently selected substituents, such as halo, hydroxyl, amino, alkyl, or alkoxy, alkylsulfony, cyano, nitro, and the like.

The term “heteroaryl” or “aromatic heterocycle” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” may also include ring systems having one or two rings wherein at least one of the rings is heteroaromatic, e.g., the other cyclic rings can be cycloalkyl, cycloalkenyl, cycloalkynyl, aromatic carbocycle, heteroaryl, and/or heterocycle. Heteroaryl groups include, for example, pyrrole, furan, thiophene, imidazole, oxazole, thiazole, pyrazole, pyridine, pyrazine, pyridazine, and pyrimidine.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alkyl and dialkylamino, acylamino, thio, and the like, and combinations thereof.

The term “optionally substituted,” or “optional substituents,” as used herein, means that the groups in question are either unsubstituted or substituted with one or more of the substituents specified. When the groups in question are substituted with more than one substituent, the substituents may be the same or different. Furthermore, when using the terms “independently,” “independently are,” and “independently selected from” mean that the groups in question may be the same or different. Certain of the herein defined terms may occur more than once in the structure, and upon such occurrence each term shall be defined independently of the other.

The term “prodrug” refers to bioreversible derivatives of drug molecules that undergo an enzymatic and/or chemical transformation in vivo to release the active parent drug, which can then exert the desired pharmacological effect. The prodrug strategy can improve the physicochemical, biopharmaceutical, or pharmacokinetic properties of pharmacologically potent compounds, and thereby enhance the development and usefulness of a potential drug product.

The term “patient” includes human and non-human animals such as companion animals (dogs and cats and the like) and livestock animals. Livestock animals are animals raised for food production. The patient to be treated is preferably a mammal, in particular a human being.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

It is to be understood that the total daily usage of the compounds and compositions described herein may be decided by the attending physician within the scope of sound medical judgment. The specific therapeutically effective dose level for any particular patient will depend upon a variety of factors, including the disorder being treated and the severity of the disorder; activity of the specific compound employed; the specific composition employed; the age, body weight, general health, gender, and diet of the patient: the time of administration, and rate of excretion of the specific compound employed, the duration of the treatment, the drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosage may be single or divided, and may be administered according to a wide variety of dosing protocols, including q.d., b.i.d., t.i.d., or even every other day, once a week, once a month, and the like. In each case the therapeutically effective amount described herein corresponds to the instance of administration, or alternatively to the total daily, weekly, or monthly dose.

As used herein, the term “therapeutically effective amount” refers to that amount of active compound or pharmaceutical agent that elicits the biological or medicinal response in a tissue system, animal or human that is being sought by a researcher, veterinarian, medical doctor or other clinicians, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment.

As used herein, the term “therapeutically effective amount” refers to the amount to be administered to a patient, and may be based on body surface area, patient weight, and/or patient condition. In addition, it is appreciated that there is an interrelationship of dosages determined for humans and those dosages determined for animals, including test animals (illustratively based on milligrams per meter squared of body surface) as described by Freireich, E. J., et al., Cancer Chemother. Rep. 1966, 50 (4), 219, the disclosure of which is incorporated herein by reference. Body surface area may be approximately determined from patient height and weight (see, e.g., Scientific Tables, Geigy Pharmaceuticals, Ardley, N.Y., pages 537-538 (1970)). A therapeutically effective amount of the compounds described herein may be defined as any amount useful for inhibiting the growth of (or killing) a population of malignant cells or cancer cells, such as may be found in a patient in need of relief from such cancer or malignancy. Typically, such effective amounts range from about 5 mg/kg to about 500 mg/kg, from about 5 mg/kg to about 250 mg/kg, and/or from about 5 mg/kg to about 150 mg/kg of compound per patient body weight. It is appreciated that effective doses may also vary depending on the route of administration, optional excipient usage, and the possibility of co-usage of a compound with other conventional and non-conventional therapeutic treatments, including other anti-tumor agents, radiation therapy, and the like.

The present invention may be better understood in light of the following non-limiting compound examples and method examples.

In one illustrative embodiment, disclosed herein are compounds having the formula (I):

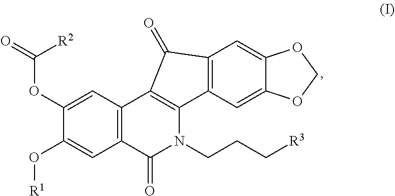

or a pharmaceutically acceptable salt, hydrate, or solvate thereof, wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
R² is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₈ cycloalkylamide, C₁-C₆ alkylamide, C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
and
R³ is

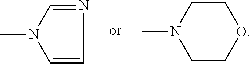

In some embodiments, disclosed herein are compounds having the formula

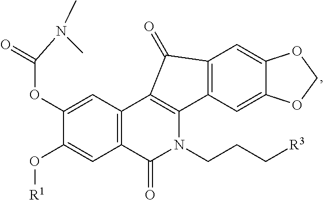

wherein:
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
and
R³ is

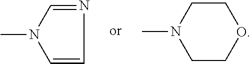

In some embodiments, disclosed herein are compounds having the formula:

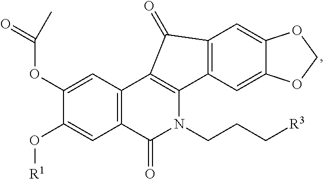

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

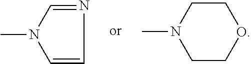

In some embodiments, disclosed herein are compounds having the formula:

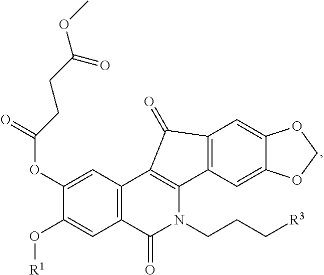

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

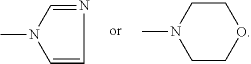

In some embodiments, disclosed herein are compounds having the formula:

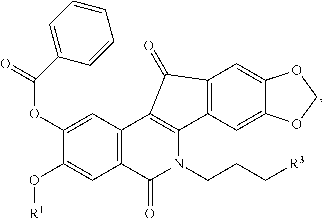

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

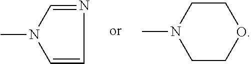

In some embodiments, disclosed herein are compounds having the formula:

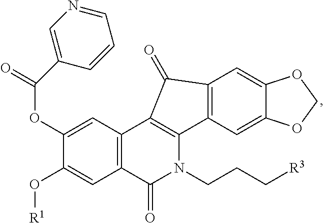

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

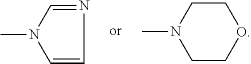

In one illustrative embodiment, disclosed herein are compounds having the formula:

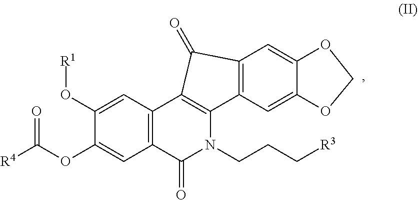

or a pharmaceutically acceptable salt, hydrate, or solvate thereof, wherein:
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
R³ is

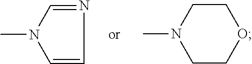

and
R⁴ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₈ cycloalkylamide, C₁-C₆ alkylamide, C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl.

In some embodiments, disclosed herein are compounds having the formula:

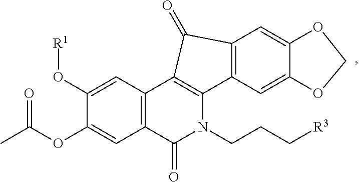

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

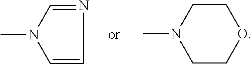

In some embodiments, disclosed herein are compounds having the formula:

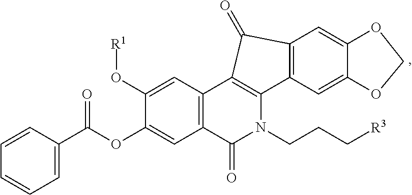

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

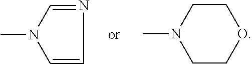

In some embodiments, disclosed herein are compounds having the formula:

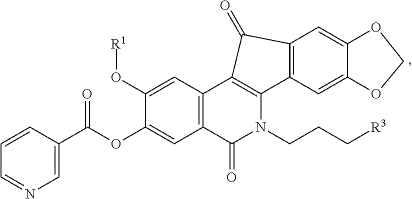

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
and
R³ is

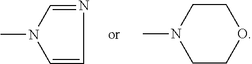

In some embodiments, disclosed herein are compounds having the formula:

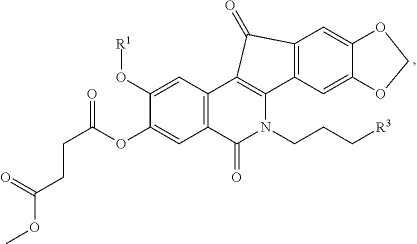

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

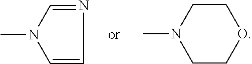

In some embodiments, disclosed herein are compounds having the formula:

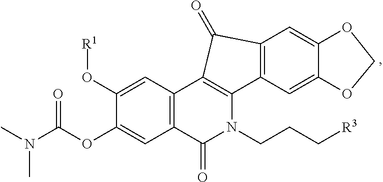

wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

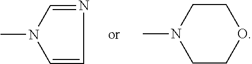

In another illustrative embodiment, disclosed herein are compounds having the formula:

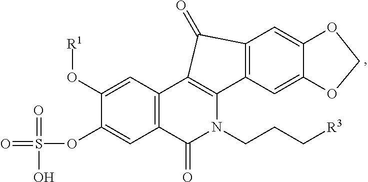

or a pharmaceutically acceptable salt, hydrate, or solvate thereof, wherein

R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

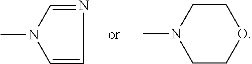

In one embodiment, disclosed herein is a pharmaceutical composition comprising a compound of formula (I), and one or more pharmaceutically acceptable carriers, diluents, and excipients.

In another embodiment, disclosed herein is a pharmaceutical composition comprising a compound of formula (II), and one or more pharmaceutically acceptable carriers, diluents, and excipients.

In another embodiment, disclosed herein is a pharmaceutical composition comprising a compound of formula (III), and one or more pharmaceutically acceptable carriers, diluents, and excipients.

In another embodiment, disclosed herein is a pharmaceutical composition comprising a compound of formula (I), (II), or (IIII), in combination with one or more other therapeutically active compounds by the same or different mode of action, and one or more pharmaceutically acceptable carriers, diluents, and excipients.

It is to be understood that the pharmaceutical composition described herein may include other components, including, but not limited to, other therapeutically active compounds by the same or different mode of action, and one or more pharmaceutically acceptable carriers, diluents, excipients, and the like.

In another embodiment, pharmaceutical compositions described herein may contain two or more of the compounds disclosed in this invention.

In one embodiment, disclosed herein is a method for treating a patient with cancer, the method comprising the step of administering a therapeutically effective amount of a pharmaceutical composition comprising the compound of formula (I) to the patient in need of relief from said cancer. It is to be understood that the composition may include other components, including, but not limited to, other therapeutically active compounds, and one or more pharmaceutically acceptable carriers, diluents, excipients, and the like.

In one embodiment, disclosed herein is a method for treating a patient with cancer, the method comprising the step of administering a therapeutically effective amount of a pharmaceutical composition comprising the compound of formula (II) to the patient in need of relief from said cancer.

In one embodiment, disclosed herein is a method for treating a patient with cancer, the method comprising the step of administering a therapeutically effective amount of a pharmaceutical composition comprising the compound of formula (III) to the patient in need of relief from said cancer.

In another embodiment, disclosed herein is a method for treating a patient with cancer, the method comprising the step of administering a therapeutically effective amount of a compound of the formula (I), (II), or (III), in combination with one or more therapeutically effective compounds by the same or different mode of action to the patient in need of relief from said cancer.

In some embodiments, it is appreciated herein that the compounds described herein may be used in combination with other compounds that are administered to treat other symptoms of cancer, such as compounds administered to relieve nausea, vomiting, pain, osteoporosis, and the like.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

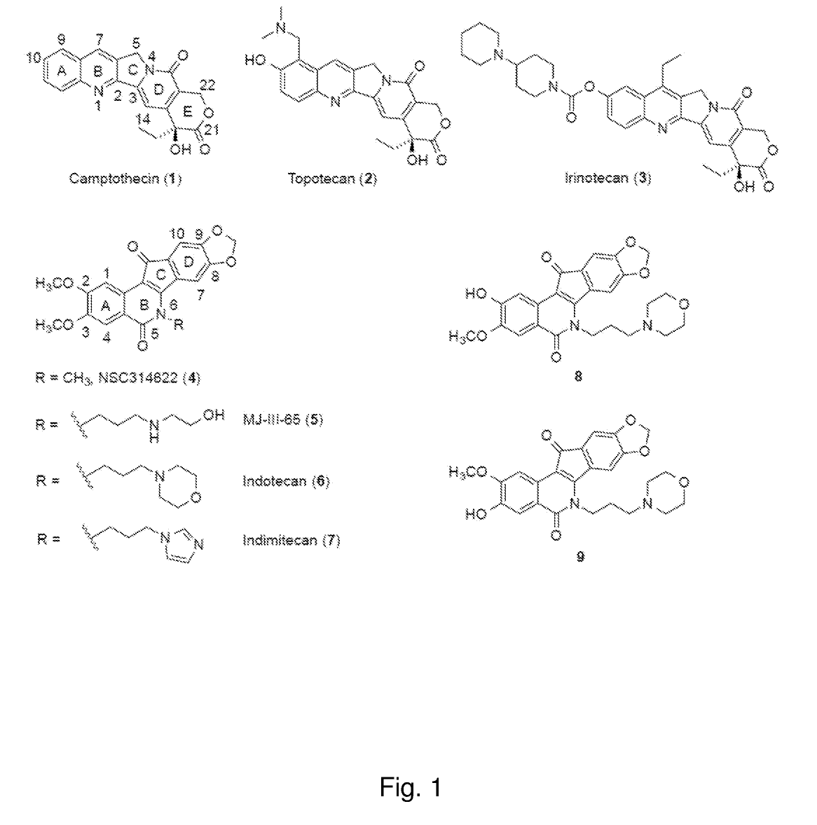

A molecular docking study was performed using the crystal structure (PDB ID: 1SC7) of a Top1-DNA-indenoisoquinoline ternary cleavage complex (Staker, B. L. et al., J. Med. Chem. 2005, 48, 2336-2345) in order to guide structural modifications of the inhibitor as well as to help understand the Top1 inhibition results. The energy-minimized structure of the morpholine derivative 8 (FIG. 1) was docked into the crystal structure of a Top1-DNA cleavage site with GOLD using the centroid coordinates of the indenoisoquinoline ligand. Compound 8 intercalates readily at the DNA cleavage site, between the +1 and −1 base pairs. Rings A and B stack with the scissile strand bases, while rings C and D stack with the noncleaved strand bases. The carbonyl group on the C ring forms a hydrogen bond to a nitrogen of the Arg364 side chain with an N—O distance of 2.5 Å, which is an essential contact for the Top1 inhibitory activity (Cinelli, M. A., et al., J. Med. Chem. 2012, 55, 10844-10862).

The previously reported crystal structure of an analogue of 4 having a 3′-carboxypropyl substituent on the lactam nitrogen in ternary complex with DNA and Top1 indicates that the A ring of the indenoisoquinoline is next to the cleaved DNA strand, where there is more room to accommodate substituents on the drug. Therefore, a series of 0-2 modified indenoisoquinoline derivatives was designed and synthesized with 2-OH indenoisoquinoline 8 as the starting material. At the same time, a series of O-3 modified indenoisoquinoline derivatives was also prepared in order to obtain O-3 modified indenoisoquinoline prodrugs that would allow a comprehensive structure-activity relationship analysis of the A-ring-modified indenoisoquinolines.

The synthesis of indenoisoquinoline 8 was performed according to the previously reported method with some modifications. With the 2-hydroxylated indenoisoquinoline 8 in hand, carbamate 10 was first prepared as shown in Scheme 2. Subsequently, four different esters were prepared. The acetylated derivative 11 was obtained by reacting the phenol 8 with acetic anhydride in the presence of DMAP. Treatment of compound 8 with methyl 4-chloro-4-oxobutyrate in the presence of DMAP provided compound 12. Reaction of compound 8 with benzoyl chloride in the presence of DMAP yielded compound 13. The nicotinate ester 14 was prepared by EDC coupling compound 8 and nicotinic acid in the presence of DMAP.

At the same time, a series of O-3-modified indenoisoquinolines 15-19 were synthesized (Scheme 3) by similar methods as those used for compounds 10-14. The sulfate compound 20, which is a potential metabolite of indotecan, was prepared by treatment of compound 9 with SO₃.NMe₃ complex in refluxing anhydrous acetonitrile.

Scheme 2^{a} Synthesis of compounds 10-14

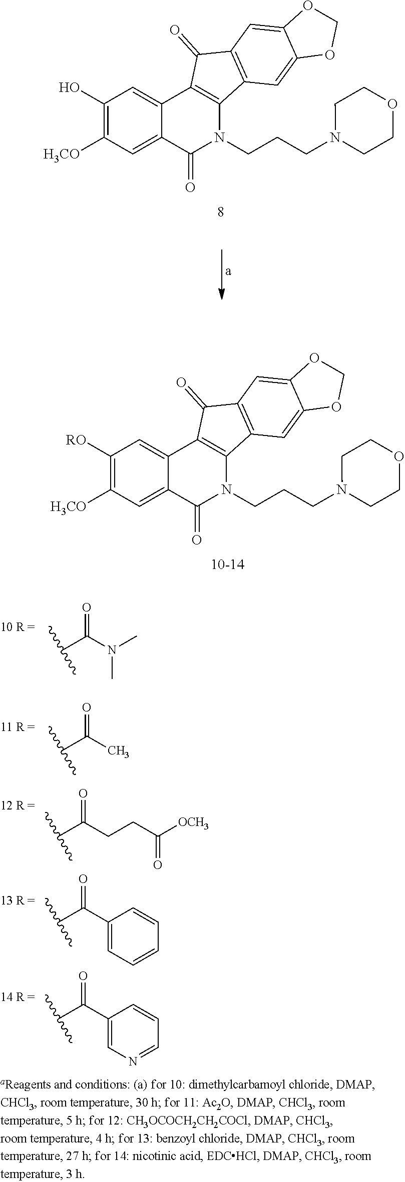

Scheme 3^{a} Synthesis of compounds 15-20

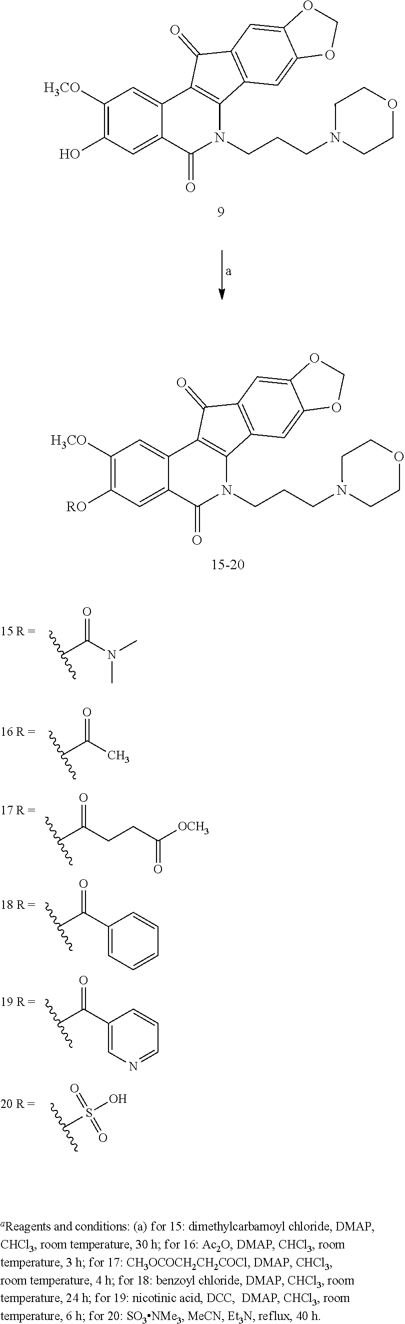

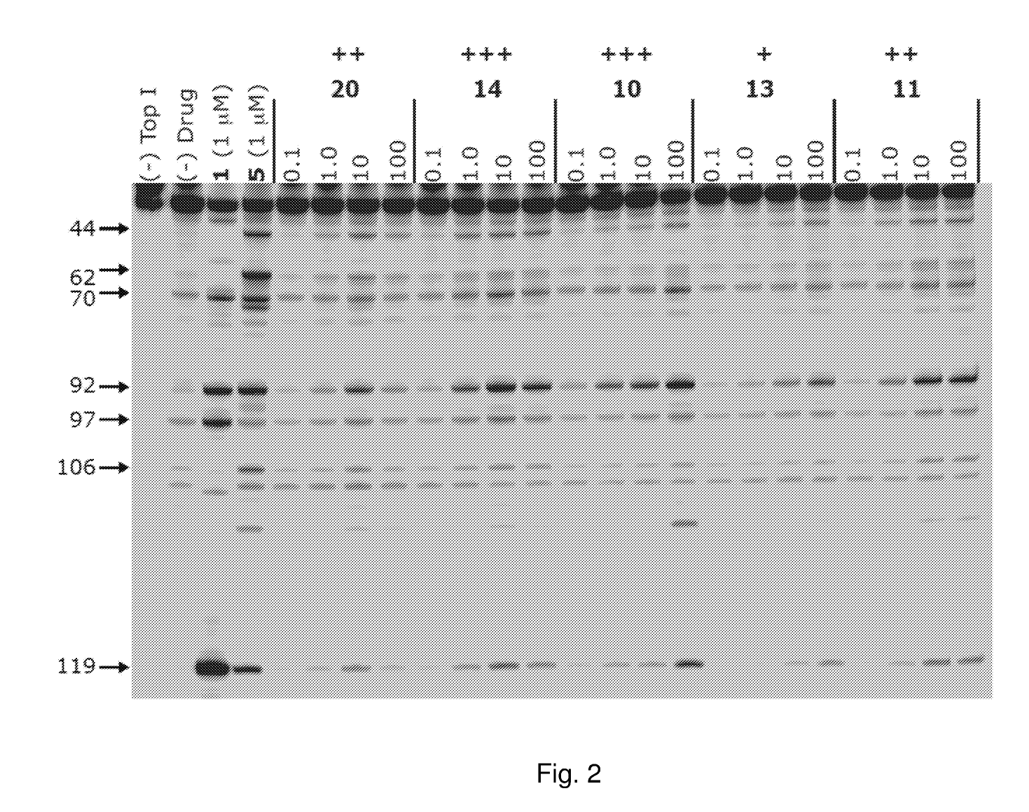

All of the new indenoisoquinoline derivatives were tested in Top1-mediated DNA cleavage assays. For this purpose, a ³²P 3′-end labeled 117-bp DNA fragment was incubated with Top1 at four concentrations of a tested compound. The DNA fragments were separated on 20% PAGE denaturing gels. The Top1 inhibitory activities were assigned on the basis of the visual inspection of the number and intensities of the gel bands corresponding to Top1-mediated DNA cleavage fragments and scored relative to the Top1 inhibitory activity of compounds 1 and 4. Results are expressed in semiquantitative fashion: 0, no detectable activity; +, weak activity; ++, similar activity to compound 4; +++, greater activity than 4; ++++, equipotent with 1. Ambiguous scores (e.g., between two values) are designated with parentheses (e.g., ++(+) would be between ++ and +++). Representative PAGE results are shown in FIG. 2. As shown in Table 1, the 2-OH indenoisoquinoline 8 had good Top1 inhibitory activity at the ++(+) level. After conversion of the hydroxyl to a dimethylcarbamate, the observed Top1 inhibitory activity increased from ++(+) for compound 8 to +++ for compound 10. Introduction of an acetyl group at the 0-2 position yielded compound 11, which was also a Top1 poison with activity at the ++ level. Subsequently, several hydrophobic substituents were attached to the 0-2 position. Both compounds 12 and 13, which have an aliphatic and aromatic substituent on the 0-2 position, respectively, displayed reduced Top1 inhibitory activity at the + level. Compound 14, which has a nicotinoyl moiety on the 0-2 position, was found to be a promising Top1 poison with activity at the +++ level.

The Top1 inhibitory activities of O-3-modified indenoisoquinolines 16-20 were found to be either abolished or significantly reduced relative to the phenol 9, which indicates that these derivatives are likely to be functioning as prodrugs since they maintain cytotoxicity despite having greatly diminished Top1 inhibitory activities. The two benzoates 13 and 18, on the other hand, were significantly less cytotoxic than their respective parent compounds, and they are both weak Top1 poisons indicating that they may not be acting as prodrugs. In general, the 0-2 modified indenoisoquinolines 10-14 are more potent Top1 poisons than their 0-3 modified counterparts 15-19.

The Top1-mediated DNA fragmentation patterns produced by camptothecin (1), indenoisoquinoline 5, and compounds 10, 11, 13, 14 and 20 are presented in FIG. 2. The sequence preferences for trapping the Top1-DNA cleavage complexes by these indenoisoquinolines are similar to each other, but the pattern is different from camptothecin, indicating that the indenoisoquinolines target the genome differently than camptothecin.

TABLE 1
Antiproliferative Potencies and Topoisomerase I Inhibitory Activities of A-ring Modified indenoisoquinolines

| | Cytotoxicity (GI₅₀ in μM)^{a} | | | | | | | | | |
|---|---|---|---|---|---|---|---|---|---|---|
| | lung | Colon | CNS | melanoma | Ovarian | renal | prostate | breast | | Top 1 |
| Compd. | HOP-62 | HCT-116 | SF-539 | UACC-62 | OVCAR-3 | SN12C | DU-145 | MCF-7 | MGM^{b} | Cleavage^{c} |
| 8 | 0.257 | 0.279 | 0.335 | 0.282 | 0.871 | 0.195 | 0.257 | 0.144 | 0.412 ± 0.005^{d} | ++(+) |
| 10 | 1.698 | 6.067 | 1.862 | 4.467 | 12.022 | 2.399 | 1.549 | 0.022 | 3.461 ± 0.999 | +++ |
| 11 | 0.018 | 0.029 | <0.01 | 0.018 | 0.138 | <0.01 | <0.01 | <0.01 | 0.043 ± 0.019 | ++ |
| 12 | <0.01 | 0.014 | <0.01 | <0.01 | 0.066 | <0.01 | <0.01 | <0.01 | 0.049 ± 0.008 | + |
| 13 | 0.079 | 0.85 | 0.037 | 0.096 | 4.57 | 0.054 | 0.051 | 0.033 | 1.5 ± 0.17 | + |
| 14 | 0.012 | 0.030 | <0.01 | 0.022 | 0.295 | <0.01 | 0.013 | <0.01 | 0.158 ± 0.065 | +++ |
| 9 | 0.01 | 0.02 | <0.01 | <0.01 | 0.03 | <0.01 | <0.01 | <0.01 | 0.076 ± 0.028 | +++(+) |
| 15 | 0.032 | 0.026 | 0.021 | 0.017 | 0.105 | 0.025 | 0.032 | <0.01 | 0.070 ± 0.028 | ++(+) |
| 16 | <0.01 | <0.01 | <0.01 | <0.01 | 0.269 | <0.01 | <0.01 | <0.01 | 0.095 ± 0.045 | 0 |
| 17 | 0.016 | 0.020 | <0.01 | 0.018 | 0.037 | <0.01 | 0.014 | <0.01 | 0.036 ± 0.018 | 0 |
| 18 | 2.14 | 5.89 | 0.135 | 0.059 | 30.903 | 0.195 | 0.724 | 0.015 | 3.19 ± 0.622 | + |
| 19 | <0.01 | <0.01 | <0.01 | <0.01 | 0.015 | <0.01 | <0.01 | <0.01 | 0.035 ± 0.000 | + |
| 20 | 0.389 | 0.389 | 0.263 | 0.263 | 1.122 | 0.355 | 0.417 | 0.209 | 0.912 | ++ |

^{a}The cytotoxicity GI₅₀ values are the concentrations corresponding to 50% growth inhibition.
^{b}Mean graph midpoint for growth inhibition of all human cancer cell lines successfully tested, ranging from 10⁻⁸ to 10⁻⁴ molar.
^{c}Compound-induced DNA cleavage due to Top1 inhibition is graded by the following rubric relative to 1 μM camptothecin: 0, no inhibitory activity; +, between 20 and 50% activities; ++, between 50 and 75% activity; +++, between 75 and 95% activity; ++++, equipotent.
^{d}For MGM GI₅₀ values in which a standard error appears, the GI₅₀ values for individual cell lines are the average of two determinations; values without standard error are from one determination.

All of the indenoisoquinolines were tested for antiproliferative activity in the National Cancer Institute's 60 cell line screen (NCI60). The cells were incubated with the tested compounds at 100, 10, 1, 0.1, and 0.01 μM concentrations for 48 h before treatment with sulforhodamine B dye. Optical densities were recorded, and their ratios relative to that of the control were plotted as percentage growth against the log 10 of the tested compound concentrations. The concentration that corresponds to 50% growth inhibition (GI₅₀) is calculated by interpolation between the points located above and below the 50% percentage growth. The mean-graph midpoint (MGM) is an estimated average of the GI₅₀ values derived from the NCI collection of 60 human cancer cell lines, where during the MGM calculation, anticancer agents with GI₅₀ values that are outside the testing range of 0.01-100 μM are arbitrarily assigned the values of 0.01 and 100 μM, respectively. The results are listed in Table 1. Most of the new compounds display significant potencies against various cell lines with GI₅₀'s in the nanomolar range, while compounds 10, 13 and 18 are in the micromolar range. Also, the GI₅₀ values of many of the substances vs. individual human cancer cell lines are below the lowest concentration used (0.01 μM) in the standard NCI testing protocol. The nicotinoyl derivative 14 has good Top1 inhibitory activity at the +++ level as well as good anti proliferative activity with an MGM value of 0.158±0.065 M. In general there is a revealing lack of correlation between the rank order of observed cytotoxicity and inhibition of Top1. For example, the MGM cytotoxicity values of 9, 15-17, and 19 are all very close to each other, but the Top1 inhibitory activities range from +++(+) to 0. This suggests that 15-17 and 19 are hydrolyzed intracellularly to the same biologically active species 9. In the case of 8, 11, and 12, the cytotoxicity of the assumed prodrugs 11 and 12 are actually greater than the parent 8 by a factor of almost one order of magnitude, which could possibly be explained by a more effective cellular penetration by 11 and 12 vs. 8, followed by hydrolysis of 11 and 12 to 8.

In order to investigate the possibility that hydrolysis of the prodrugs takes place extracellularly rather than intracellularly, compound 17 was added to cell-free cell culture medium and the mixture was incubated at 37° C. Relatively slow conversion to the parent phenol 9 was observed, with 22% conversion after 99 hours. This result suggests that the prodrug is taken up by the cells and then hydrolyzed to the phenol during the 48-hour cell culture incubation period.

A series of indenoisoquinolines substituted in the 2- and 3-positions with ester side chains were designed and synthesized for possible deployment as prodrugs. A slight improvement in Top1 inhibitory activity was observed for compounds 10 and 14 vs. 8. The rest of the assumed prodrugs are less active vs. Top1 than their parent compounds. There is significant enhancement of the cytotoxicities of the acetate 11 and the succinate 12 vs. the parent phenol 8, which would be consistent with more favorable cellular uptake followed by metabolism to the parent indenoisoquinolines. The fact that many of the derivatives have very potent cytotoxicity of similar magnitude despite being inactive or significantly less active Top1 poisons than the parent drugs suggests that they are functioning as prodrugs of the parent compounds. These new prodrug analogues of LMP400 are novel candidates for clinical development.

### EXPERIMENTAL SECTION

General.

Solvents and reagents were purchased from commercial vendors and were used without any further purification. Melting points were determined using capillary tubes with a Mel-Temp apparatus and are uncorrected. Infrared spectra were obtained using KBr pellets. IR spectra were recorded using a Perkin-Elmer 1600 series or Spectrum One FTIR spectrometer. ¹H NMR spectra were recorded at 300 MHz using a Bruker ARX300 spectrometer with a QNP probe. ESIMS analyses were performed at the Purdue Campus-Wide Mass Spectrometry Center on a Finnegan-MATT LCQ Classic mass spectrometer.

Analytical thin layer chromatography was done on Baker-flex silica gel IB2-F plates, and compounds were visualized with short wavelength UV light and ninhydrin staining. Silica gel flash chromatography was accomplished using 230-400 mesh silica gel. HPLC analyses were completed on a Waters 1525 binary HPLC pump/Waters 2487 dual λ absorbance detector system using a 5 μM C₁₈ reverse phase column. Compound purities were estimated by reversed phase C₁₈ HPLC, with UV detector at 254 nm, and the major peak area of each tested compound was ≥95% of the combined total peak area. All yields refer to isolated compounds.

### 3-Methoxy-6-(3-morpholinopropyl)-5,12-dioxo-6,12-dihydro-5H-[1,3]dioxolo[4′,5′:5,6]indeno[1,2-c]isoquinolin-2-yl Dimethylcarbamate (10)

A solution of compound 8 (0.100 g, 0.216 mmol) in chloroform (10 mL) was treated with dimethylcarbamoyl chloride (0.050 g, 0.522 mmol) in the presence of DMAP (0.200 g). The mixture was stirred at room temperature for 30 h. The mixture was diluted to a volume of 150 mL with CHCl₃, washed with H₂O (2×50 mL) and saturated aqueous NaCl (50 mL), dried over anhydrous sodium sulfate, and concentrated. The resulting residue was purified by flash column chromatography (SiO₂, ˜40 g), eluting with 1% MeOH in CHCl₃, to yield the product as a solid (0.060 g, 52%): mp 268-270° C. (dec). IR (film) 2938, 1718, 1650, 1508, 1168, 1031, 863 cm⁻¹; ¹H NMR (CDCl₃) δ 8.28 (s, 1H), 7.73 (s, 1H), 7.38 (s, 1H), 7.26 (s, 1H), 7.04 (s, 1H), 6.06 (s, 2H), 4.57-4.52 (t, J=7.5 Hz, 2H), 3.93 (s, 3H), 3.69 (s, 4H), 3.16 (s, 3H), 3.03 (s, 3H), 2.53 (s, 6H), 2.01 (s, 2H); ESIMS m/z (rel intensity) 536 (MH⁺, 100); HRESIMS m/z (rel intensity) 536.2041 (MH⁺), calcd for C₂₈H₃₀N₃O₈ 536.2033. HPLC purity: 98.83% (C-18 reverse phase, MeOH-H₂O, 90:10).

### 3-Methoxy-6-(3-morpholinopropyl)-5,12-dioxo-6,12-dihydro-5H-[1,3]dioxolo[4′,5′:5,6]indeno[1,2-c]isoquinolin-2-yl Acetate (11)

A solution of compound 8 (0.093 g, 0.200 mmol) in chloroform (10 mL) was treated with Ac₂O (0.050 g, 0.45 mmol) in the presence of DMAP (0.200 g). The mixture was stirred at room temperature for 5 h. The mixture was diluted to a volume of 100 mL with CHCl₃, washed with H₂O (2×50 mL) and saturated aq NaCl (50 mL), dried over anhydrous sodium sulfate, and concentrated. The resulting residue was purified by flash column chromatography (SiO₂, ˜30 g), eluting with 1.25% MeOH in CHCl₃ to yield the title compound as a solid (0.052 g, 50%): mp 272-274° C. IR (film) 2948, 1770, 1661, 1514, 1379, 1029, 866 cm⁻¹; ¹H NMR (CDCl₃) δ 8.26 (s, 1H), 7.75 (s, 1H), 7.40 (s, 1H), 7.06 (s, 1H), 6.07 (s, 2H), 4.46-4.51 (t, J=7.8 Hz, 2H), 3.93 (s, 3H), 3.75 (s, 4H), 2.52 (s, 6H), 2.32 (s, 3H), 1.99 (s, 2H); ESIMS m/z (rel intensity) 507 (MH⁺, 100); HRESIMS m/z (rel intensity) 507.1761 (MH⁺), calcd for C₂₇H₂₇N₂O₈ 507.1767. HPLC purity: 96.34% (C-18 reverse phase, MeOH-H₂O, 85:15).

### 3-Methoxy-6-(3-morpholinopropyl)-5,12-dioxo-6,12-dihydro-5H-[1,3]dioxolo[4′,5′:5,6]indeno[1,2-c]isoquinolin-2-yl Methyl Succinate (12)

A solution of compound 8 (0.134 g, 0.288 mmol) in chloroform (10 mL) was treated with methyl 4-chloro-4-oxobutyrate (0.050 g, 0.333 mmol) in the presence of DMAP (0.200 g). The mixture was stirred at room temperature for 4 h. The mixture was diluted to a volume of 150 mL with CHCl₃, washed with H₂O (2×30 mL) and saturated aq NaCl (80 mL), dried over anhydrous sodium sulfate, and concentrated. The resulting residue was purified by flash column chromatography (SiO₂, ˜40 g), eluting with 1% MeOH in CHCl₃, to yield the product as a solid (0.068 g, 41%): mp 219-220° C. IR (film) 2931, 1773, 1658, 1309, 1125, 1031, 786 cm⁻¹; ¹H NMR (CDCl₃) δ 8.19 (s, 1H), 7.70 (s, 1H), 7.32 (s, 1H), 6.96 (s, 1H), 6.02 (s, 2H), 4.39-4.44 (t, J=7.8 Hz, 2H), 3.90 (s, 3H), 3.74 (s, 7H), 2.95-2.99 (t, J=7.2 Hz, 2H), 2.75-2.79 (t, J=6.9 Hz, 2H), 2.52 (s, 6H), 1.93-1.98 (m, 2H). ESIMS m/z (rel intensity) 579 (MH⁺, 100); HRESIMS m/z (rel intensity) 579.1975 (MH⁺), calcd for C₃₀H₃₁N₂O₁₀ 579.1979. HPLC purity: 99.05% (C-18 reverse phase, MeOH-H₂O, 90:10).

### 3-Methoxy-6-(3-morpholinopropyl)-5,12-dioxo-6,12-dihydro-5H-[1,3]dioxolo[4′,5′:5,6]indeno[1,2-c]isoquinolin-2-yl Benzoate (13)

A solution of compound 8 (0.94 g, 0.201 mmol) in chloroform (10 mL) was treated with benzoyl chloride (0.050 g, 0.362 mmol) in the presence of DMAP (0.200 g). The mixture was stirred at room temperature for 27 h. The mixture was diluted to a volume of 100 mL with CHCl₃, washed with H₂O (2×25 mL) and saturated aqueous NaCl (50 mL), dried over anhydrous sodium sulfate, and concentrated. The resulting residue was purified by flash column chromatography (SiO₂, ˜40 g), eluting with 2% MeOH in CHCl₃, to yield the product as a solid (0.065 g, 57%): mp 261-263° C. (dec). IR (film) 2342, 1744, 1650, 1508, 1308, 1032, 788 cm⁻¹; ¹H NMR (CDCl₃) δ 8.39 (s, 1H), 8.15-8.22 (m, 2H), 7.80 (s, 1H), 7.64-7.67 (m, 1H), 7.52-7.56 (m, 3H), 7.07 (s, 1H), 6.08 (s, 2H), 4.57-4.52 (t, J=7.2 Hz, 2H), 3.91 (s, 3H), 3.67 (s, 4H), 2.59 (s, 6H), 2.07 (s, 2H); ESIMS m/z (rel intensity) 569 (MH⁺, 100); HRESIMS m/z (rel intensity) 569.1930 (MH⁺), calcd for C₃₂H₂₉N₂O₈ 569.1924. HPLC purity: 96.56% (C-18 reverse phase, MeOH—H₂O, 80:20).

### 3-Methoxy-6-(3-morpholinopropyl)-5,12-dioxo-6,12-dihydro-5H-[1,3]dioxolo[4′,5′:5,6]indeno[1,2-c]isoquinolin-2-yl Nicotinate (14)

A solution of compound 8 (0.120 g, 0.26 mmol) in chloroform (10 mL) was treated with nicotinic acid (0.200 g, 1.62 mmol) in the presence of EDC-HCl (0.160 g, 0.83 mmol) and DMAP (270 mg). The mixture was stirred at room temperature for 3 h. The mixture was diluted to a volume of 120 mL with CHCl₃, washed with H₂O (2×60 mL) and saturated aqueous NaCl (60 mL), dried over anhydrous sodium sulfate, and concentrated. The resulting residue was purified by flash column chromatography (SiO₂, ˜40 g), eluting with 1.25% MeOH in CHCl₃, to yield the title compound as a solid (0.066 g, 44%): mp 277-279° C. (dec). IR (film) 2956, 1750, 1508, 1381, 1272, 1116, 865 cm⁻¹; ¹H NMR (CDCl₃) δ 9.42 (s, 1H), 8.89 (s, 1H), 8.47-8.49 (d, J=8.1 Hz, 1H), 8.41 (s, 1H), 7.81 (s, 1H), 7.47-7.52 (m, 1H), 7.41 (s, 1H), 7.08 (s, 1H), 6.09 (s, 2H), 4.51-4.56 (t, J=6.9 Hz, 2H), 3.92 (s, 3H), 3.67 (s, 4H), 2.58 (s, 6H), 2.07 (s, 2H); ESIMS m/z (rel intensity) 570 (MH⁺, 100); HRESIMS m/z (rel intensity) 570.1872 (MH⁺), calcd for C₃₁H₂₈N₃O₈ 570.1876. HPLC purity: 95.84% (C-18 reverse phase, MeOH, 100).

### 2-Methoxy-6-(3-morpholinopropyl)-5,12-dioxo-6,12-dihydro-5H-[1,3]dioxolo[4′,5′:5,6]indeno[1,2-c]isoquinolin-3-yl Dimethylcarbamate (15)

A solution of compound 9 (0.100 g, 0.216 mmol) in chloroform (15 mL) was treated with dimethylcarbamoyl chloride (0.031 g, 0.324 mmol) in the presence of DMAP (0.015 g). The mixture was stirred at room temperature for 30 h. The mixture was diluted to a volume of 100 mL with CHCl₃, washed with H₂O (2×50 mL) and saturated aqueous NaCl (50 mL), dried over anhydrous sodium sulfate, and concentrated. The resulting residue was purified by flash column chromatography (SiO₂, ˜40 g), eluting with 2.5% MeOH in CHCl₃, to yield the product (0.081 g, 70%): mp 290-292° C. IR (film) 2960, 2350, 1870, 1732, 1508, 1163, 1032, 863 cm⁻¹; ¹H NMR (CDCl₃) δ 8.01 (s, 1H), 7.93 (s, 1H), 7.26 (s, 1H), 6.82 (s, 1H), 6.09 (s, 2H), 4.50 (m, 2H), 3.98 (s, 3H), 3.82 (m, 6H), 3.14 (s, 3H), 3.02 (s, 3H), 2.61 (m, 4H), 2.05 (m, 2H); ESIMS m/z (rel intensity) 536 (MH⁺, 100); HRESIMS m/z (rel intensity) 536.2037 (MH⁺), calcd for C₂₈H₃₀N₃O₈ 536.2033. HPLC purity: 95.63% (C-18 reverse phase, MeOH-H₂O, 90:10).

### 2-Methoxy-6-(3-morpholinopropyl)-5,12-dioxo-6,12-dihydro-5H-[1,3]dioxolo[4′,5′:5,6]indeno[1,2-c]isoquinolin-3-yl Acetate (16)

A solution of compound 9 (0.047 g, 0.100 mmol) in chloroform (20 mL) was treated with Ac₂O (0.016 g, 0.15 mmol) in the presence of DMAP (0.025 g, 0.200 mmol). The mixture was stirred at room temperature for 3 h. The mixture was diluted to a volume of 100 mL with CHCl₃, washed with H₂O (2×50 mL) and saturated aqueous NaCl (50 mL), dried over anhydrous sodium sulfate, and concentrated. The resulting residue was purified by flash column chromatography (SiO₂, ˜30 g), eluting with 1.25% MeOH in CHCl₃ to yield the title compound (0.035 g, 68%): mp>350° C. IR (film) 2346, 1773, 1656, 1508, 1308, 1115, 670 cm⁻¹; ¹H NMR (CDCl₃) δ 8.10 (s, 1H), 7.90 (s, 1H), 7.43 (s, 1H), 7.08 (s, 1H), 6.09 (s, 2H), 4.49 (s, 2H), 3.98 (s, 3H), 3.80 (s, 4H), 2.58 (s, 6H), 2.34 (s, 3H), 2.05 (m, 2H); ESIMS m/z (rel intensity) 507 (MH⁺, 100); HRESIMS m/z (rel intensity) 507.1772 (MH⁺), calcd for C₂₇H₂₇N₂O₈ 507.1767. HPLC purity: 95.26% (C-18 reverse phase, MeOH-H₂O, 90:10).

### 2-Methoxy-6-(3-morpholinopropyl)-5,12-dioxo-6,12-dihydro-5H-[1,3]dioxolo[4′,5′:5,6]indeno[1,2-c]isoquinolin-3-yl Methyl Succinate (17)

A solution of compound 9 (0.060 g, 0.129 mmol) in chloroform (10 mL) was treated with methyl 4-chloro-4-oxobutyrate (0.050 g, 0.333 mmol) in the presence of DMAP (0.100 g). The mixture was stirred at room temperature for 4 h. The mixture was diluted to a volume of 100 mL with CHCl₃, washed with H₂O (2×25 mL) and saturated aqueous NaCl (50 mL), dried over anhydrous sodium sulfate, and concentrated. The resulting residue was purified by flash column chromatography (SiO₂, ˜40 g), eluting with 1.25% MeOH in CHCl₃, to yield the product as a solid (0.039 g, 53%): mp>350° C. IR (film) 2348, 1717, 1650, 1508, 1116, 670 cm⁻¹; ¹H NMR (CDCl₃) δ 8.05 (s, 1H), 7.89 (s, 1H), 7.42 (s, 1H), 7.04 (s, 1H), 6.07 (s, 2H), 4.43-4.48 (t, J=7.5 Hz, 2H), 3.96 (s, 3H), 3.76 (s, 4H), 3.65 (s, 3H), 2.93-2.97 (t, J=6.9 Hz, 2H), 2.74-2.78 (t, J=6.6 Hz, 2H), 2.53 (s, 6H), 1.99 (s, 2H); ESIMS m/z (rel intensity) 579 (MH⁺, 100); HRESIMS m/z (rel intensity) 579.1974 (MH⁺), calcd for C₃₀H₃₁N₂O₁₀ 579.1979. HPLC purity: 97.00% (C-18 reverse phase, MeOH-H₂O, 80:20).

### 2-Methoxy-6-(3-morpholinopropyl)-5,12-dioxo-6,12-dihydro-5H-[1,3]dioxolo[4′,5′:5,6]indeno[1,2-c]isoquinolin-3-yl Benzoate (18)

A solution of compound 9 (0.85 g, 0.183 mmol) in chloroform (15 mL) was treated with benzoyl chloride (0.038 g, 0.275 mmol) in the presence of DMAP (0.044 g, 0.366 mmol). The mixture was stirred at room temperature for 24 h. The mixture was diluted to a volume of 50 mL with CHCl₃, washed with H₂O (2×25 mL) and saturated aqueous NaCl (50 mL), dried over anhydrous sodium sulfate, and concentrated. The resulting residue was purified by flash column chromatography (SiO₂, ˜40 g), eluting with 2.5% MeOH in CHCl₃, to yield the product (0.062 g, 61%): mp 316-318° C. IR (film) 2957, 2348, 1651, 1559, 1309, 1116, 864 cm⁻¹; ¹H NMR (CDCl₃) δ 8.20-8.27 (m, 2H), 8.14 (s, 1H), 8.03 (s, 1H), 7.63-7.70 (m, 1H), 7.50-7.56 (m, 2H), 7.44 (s, 1H), 7.08 (s, 1H), 6.09 (s, 2H), 4.50 (s, 2H), 3.99 (s, 3H), 3.80 (s, 4H), 2.58 (s, 6H), 2.05 (s, 2H); ESIMS m/z (rel intensity) 569 (MH⁺, 100); HRESIMS m/z (rel intensity) 569.1921 (MH⁺), calcd for C₃₂H₂₉N₂O₈ 569.1924. HPLC purity: 96.75% (C-18 reverse phase, MeOH-H₂O, 85:15).

### 2-Methoxy-6-(3-morpholinopropyl)-5,12-dioxo-6,12-dihydro-5H-[1,3]dioxolo[4′,5′:5,6]indeno[1,2-c]isoquinolin-3-yl Nicotinate (19)

A solution of compound 9 (0.153 g, 0.33 mmol) in chloroform (30 mL) was treated with nicotinic acid (0.061 g, 0.495 mmol) in the presence of DCC (0.075 g, 0.363 mmol) and a catalytic amount of DMAP. The mixture was stirred at room temperature for 6 h. The mixture was diluted to a volume of 100 mL with CHCl₃, washed with H₂O (2×60 mL) and saturated aqueous NaCl (60 mL), dried over anhydrous sodium sulfate, and concentrated. The resulting residue was purified by flash column chromatography (SiO₂, ˜40 g), eluting with 2.5% MeOH in CHCl₃, to yield the title compound (0.097 g, 53%): mp 274-276° C. (dec). IR (film) 2961, 1749, 1657, 1504, 1261, 1033, 864 cm⁻¹; ¹H NMR (CDCl₃) δ 9.41 (s, 1H), 8.87 (s, 1H), 8.45-8.48 (d, J=7.8 Hz, 1H), 8.16 (s, 1H), 8.04 (s, 1H), 7.46 (s, 2H), 7.10 (s, 1H), 6.11 (s, 2H), 4.52 (m, 2H), 3.97 (s, 3H), 3.80 (s, 4H), 2.60 (s, 6H), 2.07 (s, 2H); ESIMS m/z (rel intensity) 570 (MH⁺, 100); HRESIMS m/z (rel intensity) 570.1884 (MH⁺), calcd for C₃₁H₂₇N₃O₈ 570.1876. HPLC purity: 95.53% (C-18 reverse phase, MeOH-H₂O, 85:15).

### 2-Methoxy-6-(3-morpholinopropyl)-5,12-dioxo-6,12-dihydro-5H-[1,3]dioxolo[4′,5′:5,6]indeno[1,2-c]isoquinolin-3-yl Hydrogen Sulfate (20)

A mixture of SO₃—NMe₃ (0.28 g, 2.016 mmol) and Et₃N (0.77 mL, 4.032 mmol) was added to a well-stirred mixture of compound 9 (0.156 g, 0.336 mmol) in anhydrous MeCN (8 mL) at room temperature under argon. The reaction mixture was heated at reflux under argon for 40 h. The reaction mixture was cooled to room temperature, decanted, and concentrated under reduced pressure. The crude product was applied twice to a column of silica gel (eluent 1-20% MeOH—CHCl₃). The fractions containing the target compound were concentrated, and the target compound 20 (0.094 g, 52%) was obtained by preparative silica gel TLC (2 mm, CH₃OH—CHCl₃, 1:5): mp 283-285° C. (dec). ¹H NMR (DMSO, 300 MHz) 9.42 (s, 1H), 8.24 (s, 1H), 7.91 (s, 1H), 7.55 (s, 1H), 7.13 (s, 1H), 6.21 (s, 2H), 4.46 (m, 2H), 3.88 (s, 3H), 3.55 (s, 4H), 3.07-3.11 (m, 2H), 2.37 (s, 4H), 1.90 (m, 2H); ESIMS m/z (rel intensity) negative ion 543.1 [(M-H⁺)⁻, 100]; HRESIMS m/z (rel intensity) 543.1077 [(M-H⁺)⁻], calcd for C₂₅H₂₃N₂O₁₀S 543.1073. HPLC purity: 96.67% (C-18 reverse phase, MeOH-H₂O, 90:10).

Topoisomerase I-Mediated DNA Cleavage Reactions.

Human recombinant Top1 was purified from baculovirus as previously described (Morrell, A., et al., J. Med. Chem. 2007, 50, 2040-2048). DNA cleavage reactions were prepared as previously reported with the exception of the DNA substrate (Strumberg, D., et al., J. Med. Chem. 1999, 42, 446-457). Briefly, a 117-bp DNA oligonucleotide (Integrated DNA Technologies) encompassing the previously identified Top1 cleavage sites in the 161-bp fragment from pBluescript SK(−) phagemid DNA was employed. This 117-bp oligonucleotide contains a single 5′-cytosine overhang, which was 3′-end-labeled by fill-in reaction with [α-³²P]dGTP in React 2 buffer (50 mM Tris-HCl, pH 8.0, 100 mM MgCl₂, 50 mM NaCl) with 0.5 unit of DNA polymerase I (Klenow fragment, New England BioLabs). Unincorporated [³²P]dGTP was removed using mini Quick Spin DNA columns (Roche, Indianapolis, Ind.), and the eluate containing the 3′-end-labeled DNA substrate was collected. Approximately 2 nM radiolabeled DNA substrate was incubated with recombinant Top1 in 20 μL of reaction buffer [10 mM Tris-HCl (pH 7.5), 50 mM KCl, 5 mM MgCl₂, 0.1 mM EDTA, and 15 μg/mL BSA] at 25° C. for 20 min in the presence of various concentrations of compounds. The reactions were terminated by adding SDS (0.5% final concentration) followed by the addition of two volumes of loading dye (80% formamide, 10 mM sodium hydroxide, 1 mM sodium EDTA, 0.1% xylene cyanol, and 0.1% bromophenol blue). Aliquots of each reaction mixture were subjected to 20% denaturing PAGE. Gels were dried and visualized by using a phosphoimager and ImageQuant software (Molecular Dynamics). For simplicity, cleavage sites were numbered as previously described in the 161-bp fragment.

Stability of Compound 17 in Cell Culture Medium.

RPMI 1640 medium was purchased from Sigma-Aldrich (St. Louis, Mo.). Compound 17 (10 μM) was incubated at 37° C. with RPMI 1640 medium. After 99 h, an aliquot was taken from the incubation mixture and filtered prior to analysis using LC-MS (Waters, Germany). The flow rate was 0.75 mL/min and the eluent was recorded with a DAD at 280 nm.

Molecular Modeling.

The Top1 crystal structure for docking was prepared, and the docking protocol was validated as previously described (Strumberg, D. et al., J. Med. Chem. 1999, 42, 446-457). The ternary complex ligand centroid coordinates for docking were defined using the ligand in the Top1-DNA-MJ238 crystal structure (PDB code 1SC7) as the center of the binding pocket (x=21.3419, y=−3.9888, z=28.2163). The ligand was then deleted. Indenoisoquinolines to be modeled were constructed in SYBYL. Atom types were assigned using SYBYL atom typing. Hydrogens were added, and the ligands were minimized by the conjugate gradient method using the MMFF94s force field with MMFF94 charges, a distance-dependent dielectric function, and a 0.01 kcal mol⁻¹ Å⁻¹ energy gradient convergence criterion. Each ligand was docked into the mutant crystal structure using GOLD 3.2 with default parameters, and the coordinates were defined by the crystal structure as described above. The top four poses for each ligand were examined. The highest-ranked poses for these ligands were merged into the crystal structure, and the entire complex was subsequently subjected to minimization using a standard Powell method, the MMFF94s force field and MMFF94 charges, a distance-dependent dielectric function, and a 0.05 kcal mol⁻¹ Å⁻¹ energy gradient convergence criterion. During the energy minimization, the ligand and a 7 Å sphere surrounding the ligands were allowed to move while the structures outside this sphere were frozen in an aggregate.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected. It is intended that the scope of the present methods and apparatuses be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A compound having the formula 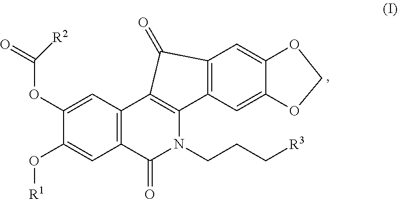
or a pharmaceutically acceptable salt, hydrate, or solvate thereof, wherein:
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
R² is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₈ cycloalkylamide, C₁-C₆ alkylamide, C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

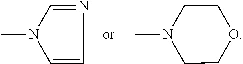

2. The compound of claim 1, wherein the compound has the formula 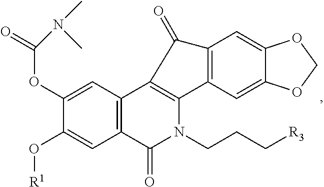
wherein: R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

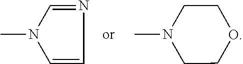

3. The compound of claim 1, wherein the compound has the formula 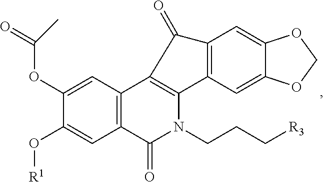
wherein R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

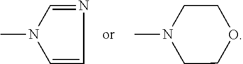

4. The compound of claim 1, wherein the compound has the formula 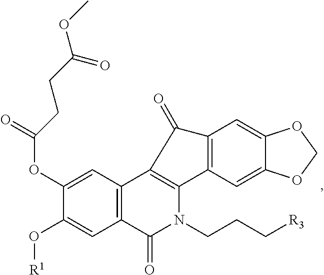
wherein R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

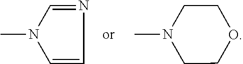

5. The compound of claim 1, wherein the compound has the formula 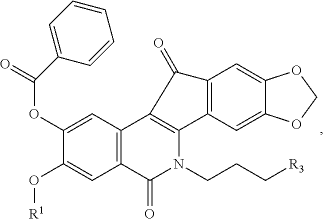
wherein R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

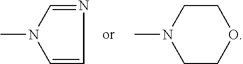

6. The compound of claim 1, wherein the compound has the formula 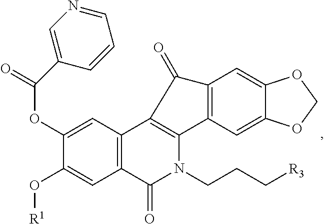
wherein R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

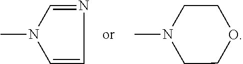

7. A compound having the formula 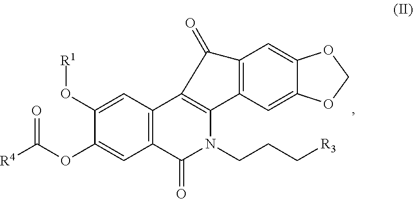
or a pharmaceutically acceptable salt, hydrate, or solvate thereof, wherein:
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl;
R³ is 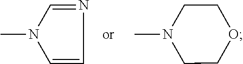
and
R⁴ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₈ cycloalkylamide, C₁-C₆ alkylamide, C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl.

8. The compound of claim 7, wherein R⁴ is methyl.

9. The compound of claim 7, wherein R⁴ is phenyl.

10. The compound of claim 7, wherein R⁴ is 3-pyrridyl.

11. The compound of claim 7, wherein R⁴ is —CH₂CH₂COOCH₃.

12. The compound of claim 7, wherein R⁴ is —N(CH₃)₂.

13. A compound having the formula 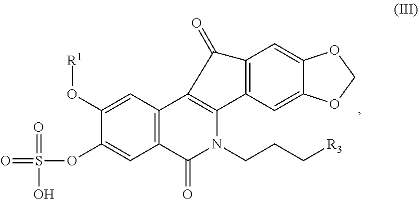
or a pharmaceutically acceptable salt, hydrate, or solvate thereof, wherein
R¹ is a C₁-C₆ alkyl, C₁-C₆ alkenyl, C₁-C₆ alkynyl, C₁-C₈ cycloalkyl, C₁-C₈ cycloalkenyl, C₁-C₈ heterocycle (heterocyclic), C₁-C₆ haloalkyl, C₁-C₆ haloalkenyl, C₁-C₆ cyanoalkyl, C₁-C₆ cyanoalkenyl, aryl, heteroaryl, optionally substituted aryl, or an optionally substituted heteroaryl; and
R³ is

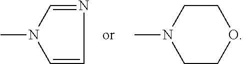

14. A pharmaceutical composition comprising the compound of claim 1, or a pharmaceutically acceptable salt thereof, together with one or more pharmaceutically acceptable carriers, diluents, and excipients.

15. A pharmaceutical composition comprising the compound of claim 7, or a pharmaceutically acceptable salt thereof, together with one or more pharmaceutically acceptable carriers, diluents, and excipients.

16. A pharmaceutical composition comprising the compound of claim 13, or a pharmaceutically acceptable salt thereof, together with one or more pharmaceutically acceptable carriers, diluents, and excipients.

17. (canceled)

18. A method for treating a patient with cancer, the method comprising the step of administering a therapeutically effective amount of the compound of claim 1 to the patient in need of relief from said cancer.

19. A method for treating a patient with cancer, the method comprising the step of administering a therapeutically effective amount of the compound of claim 7 to the patient in need of relief from said cancer.

20. A method for treating a patient with cancer, the method comprising the step of administering a therapeutically effective amount of the compound of claim 13 to the patient in need of relief from said cancer.

21. (canceled)

22. The pharmaceutical composition of claim 14 further comprising one or more other therapeutically active compounds by the same or different mode of action.

23. The pharmaceutical composition of claim 15 further comprising one or more other therapeutically active compounds by the same or different mode of action.

24. The pharmaceutical composition of claim 16 further comprising one or more other therapeutically active compounds by the same or different mode of action.

25. The method of claim 18 wherein the therapeutically effective amount of a compound of claim 1 is administered in combination with one or more therapeutically effective compounds by the same or different mode of action.

26. The method of claim 19 wherein the therapeutically effective amount of a compound of claim 7 is administered in combination with one or more therapeutically effective compounds by the same or different mode of action.

27. The method of claim 20 wherein the therapeutically effective amount of a compound of claim 13 is administered in combination with one or more therapeutically effective compounds by the same or different mode of action.
